(19) United States
(12) Patent Application Publication
(10) Pub. No.: US 2021/0361240 A1
(43) Pub. Date: Nov. 25, 2021

(54) RATI-FI EMPLOYEE MONITORING SYSTEM

(71) Applicant: Jonathan Fialkov, Urbandale, IA (US)

(72) Inventor: Jonathan Fialkov, Urbandale, IA (US)

(21) Appl. No.: 16/879,001

(22) Filed: May 20, 2020

(51) Int. Cl. A61B 5/00; G16H 10/20; G16H 10/60; G16H 50/80; G16H 50/30

(52) U.S. Cl. CPC A61B 5/6898; G16H 10/20; A61B 2503/20; G16H 50/80; G16H 50/30; G16H 10/60

(57) ABSTRACT

The present invention comprises a health screening and recording tool for use in tracking certain health metrics in a population for the purpose of evaluating the health of individuals in the population, as well as the metrics of the population, and for use in decision making regarding activities.

### BACKGROUND

The recent COVID-19 pandemic has highlighted new informational needs for monitoring and managing populations. Early in the pandemic, it was believed that the populations about which information would be helpful would include country, state, county, city, etc. As the pandemic progressed, it became clear that the virus was most often spread via close contact or, at least, aerosol contact between people. As the pandemic progressed, certain businesses were subjected to guidelines to keep patrons safe, such as one-way grocery aisles, and six-foot markers to keep people distanced from each other. Guidelines were promulgated suggesting or requiring distances of 6 feet or more between individuals due to the expected distance of travel of the virus via normal exhale; facial masks were sometimes required and often suggested.

For industries such as meat packing plants and other factories where people previously worked near one another, all day, without dividers and without masks, the spread of the virus was, on occasion, out of control. In those situations, the virus spread and sickened large portions of an employee population in a given factory or industry. These industries responded in a haphazard manner; some sent workers home only if the worker reported ill and a healthcare worker on site confirmed an illness; most did not implement pro-active protocols for detection of illness. Some—but certainly not all—of those workplaces, installed dividers and required workers to wear masks. But, despite the installation of dividers and new requirements to wear masks, new cases of the virus in workers still occurred. And, further, it was not yet known if a person would be vulnerable to the virus more than once. The shortage in work force created by the virus quickly translated to a shortage in supply of such essential items as meat.

The pandemic moved forward and we began to better understand its early symptoms, how to detect whether a person was infected and became more adept at prevention and treatment. But, the need to continue certain business endeavors even in the face of the lethality of this disease could not be ignored, and many businesses reopened, significantly increasing the debilitating an potentially lethal effect.

Simple detection and treatment were determined early. Thereafter, it was determined that the spread was mostly contact and aerosol. This information presented huge challenges to ongoing business endeavors. Then, studies began to show how long the virus survived on certain surfaces, and how far it traveled when projected by, for example, a human cough. At that point, determining ways to prevent spread of the disease became critical. With still no sure date predicted for arrival of a vaccine, employers shifted from crisis management to a more pro-active stance. In factories or other businesses where a high number of individuals work in relatively close quarters, the need for means to pre-screen a worker prior to reporting to work each day, and for means to record such pre-screening and health information for comparison and tracking, along with each employee's physical location when performing his duties has become an essential need. So, too, have become essential dividers, masks, gloves, hygiene, and disinfection of surfaces, where possible. But without continued testing and additional efforts to understand to whom and how the disease is spreading, it will be difficult to stop its spread.

There is, therefore, a strong need to provide an early and consistent means to track serial data that may be related to or otherwise be indicative of infection of a person by the virus, and combine that data with data pertaining to work location, place on the line in the factory, the identity of workers in close proximity to the person infected, etc. Early detection or even prediction of those who are likely to be infected next could provide means to pro-actively manage a workforce to maintain health and, therefore, productivity.

### BRIEF DESCRIPTION OF THE DRAWINGS

FIG. 1 Login Screen

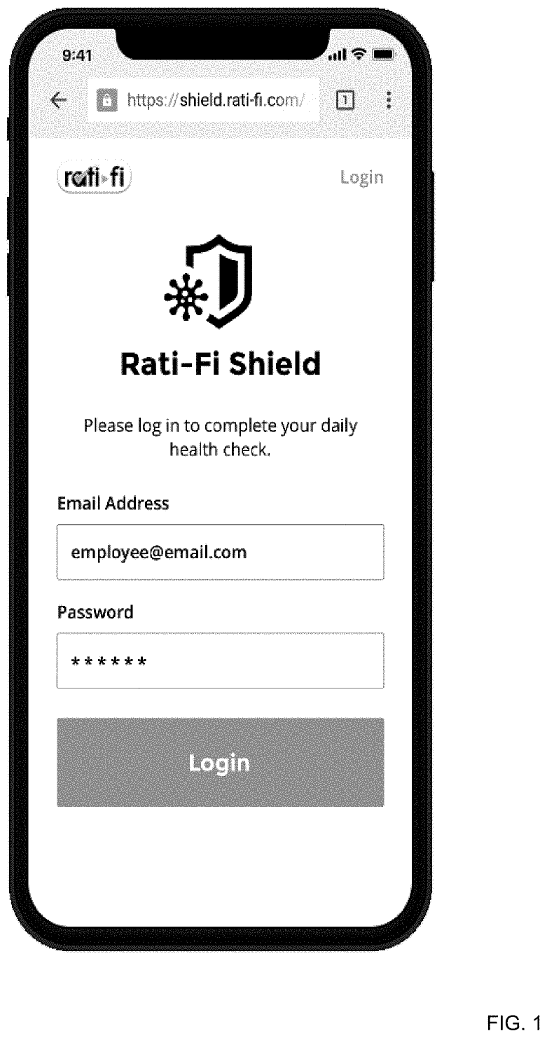

FIG. 2 Stop Before You Begin

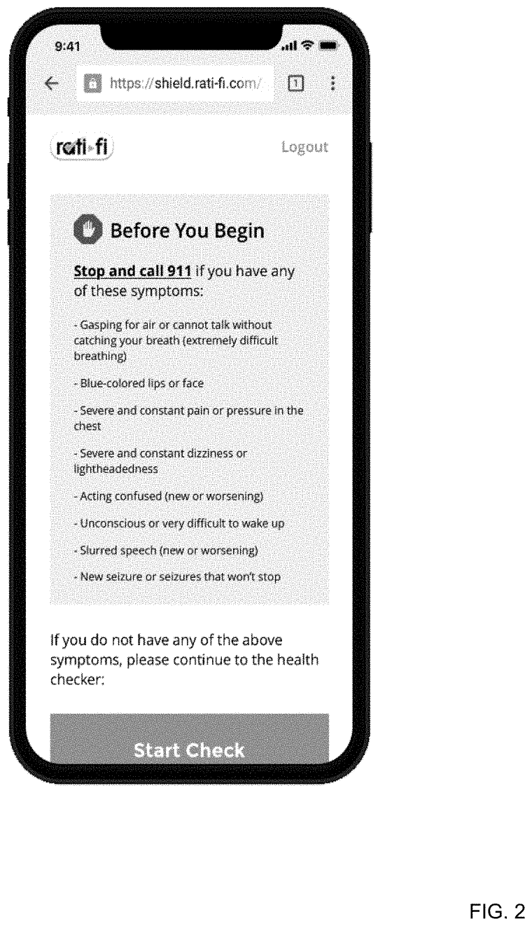

FIG. 3 Have Any of The Following?

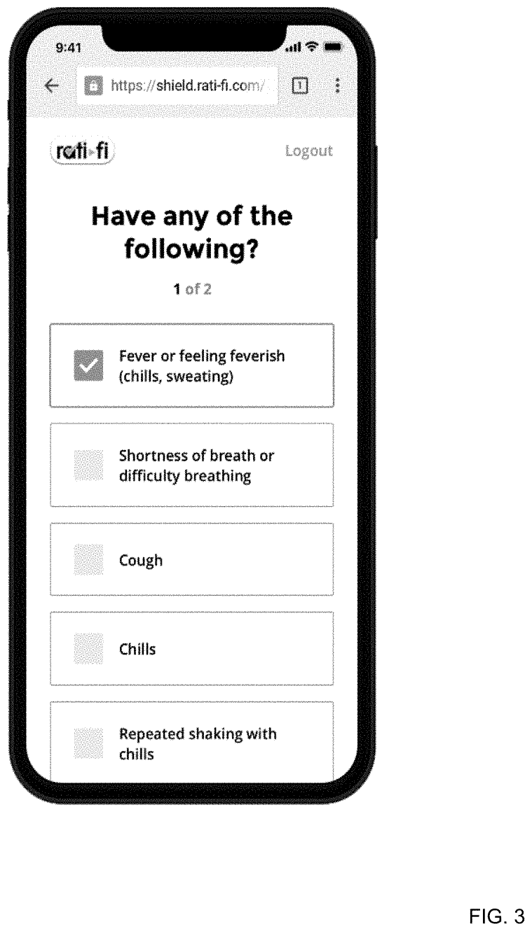

FIG. 4 Have Any of The Following?

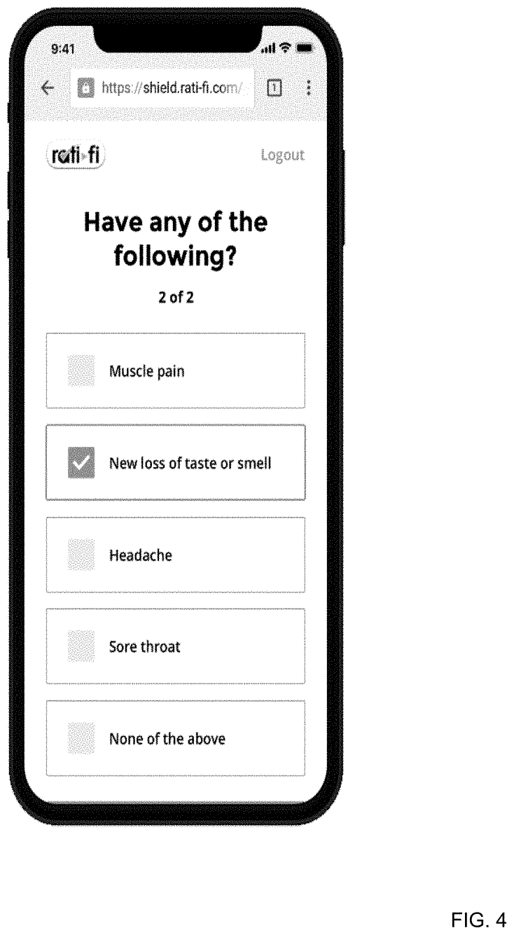

FIG. 5 Have You Had Contact With . . .

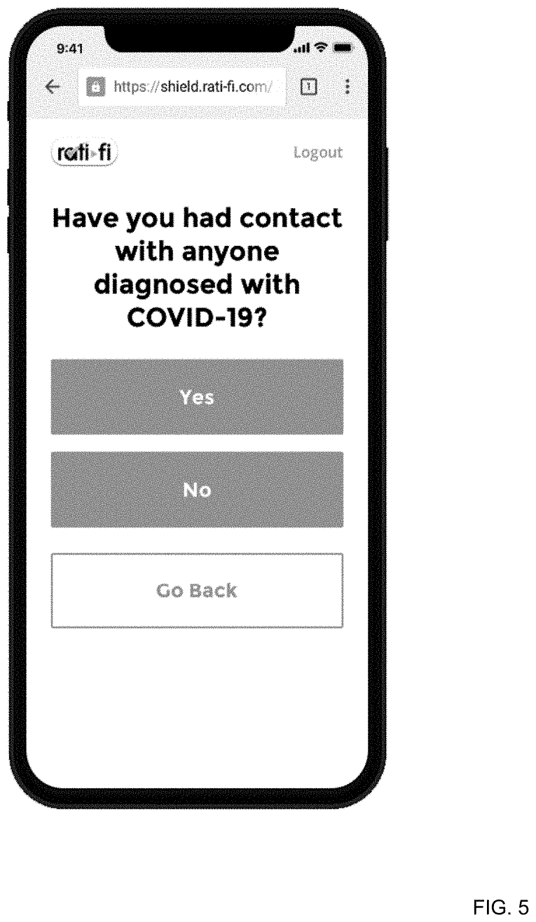

FIG. 6 Have you Traveled Outside Your City?

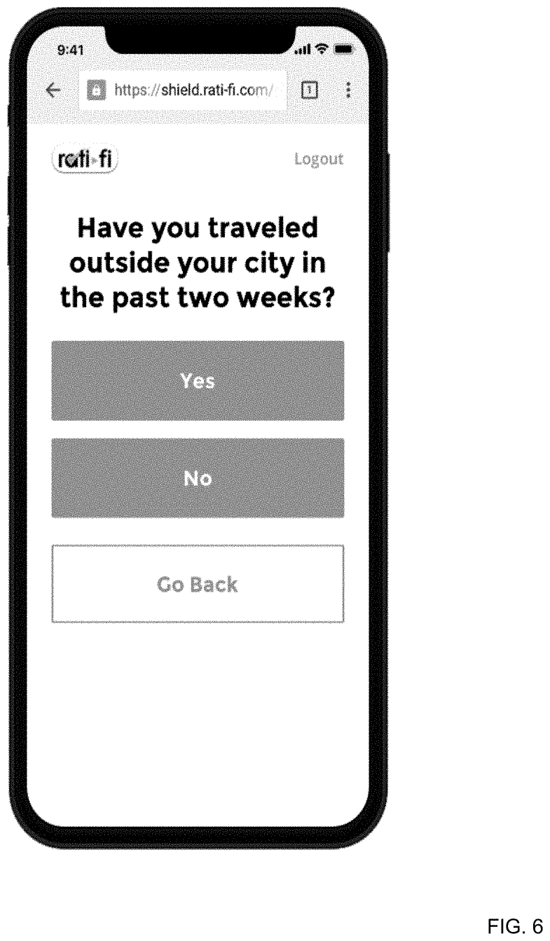

FIG. 7 Temperature Do You Have a Thermometer

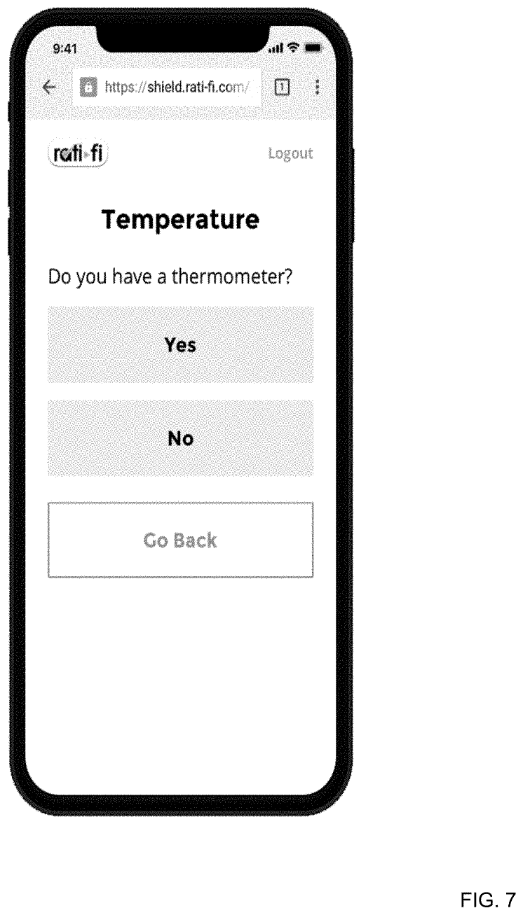

FIG. 8 Please Enter Your Current Temperature

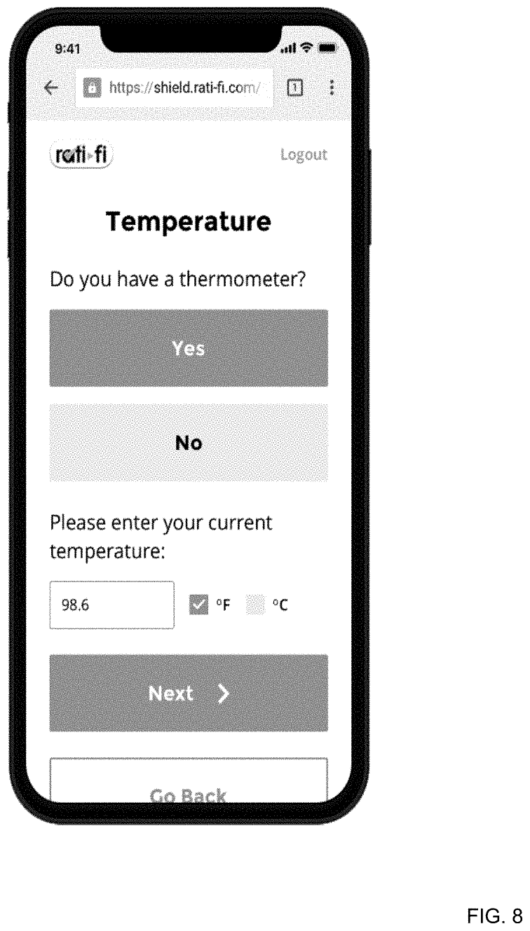

FIG. 9 Please Confirm Consent to Share Information for Testing and Tracing

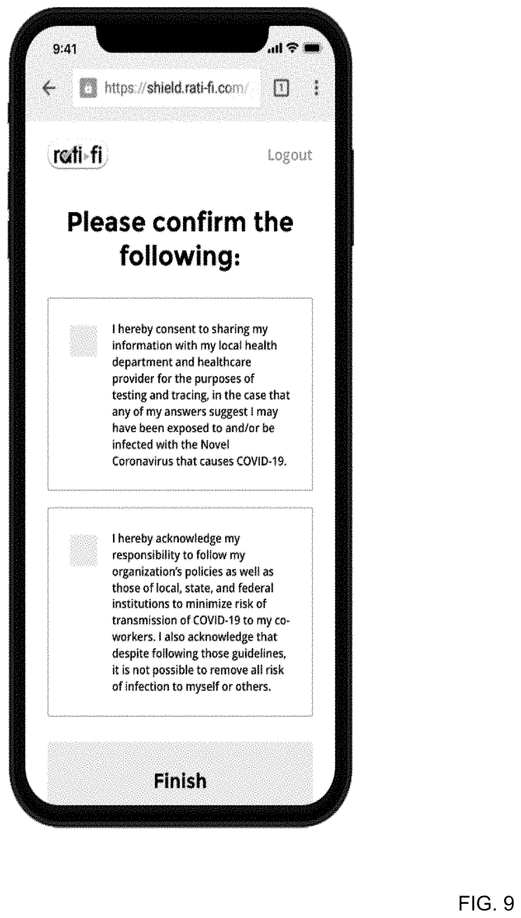

FIG. 10 Acknowledge Responsibility to Follow Policy

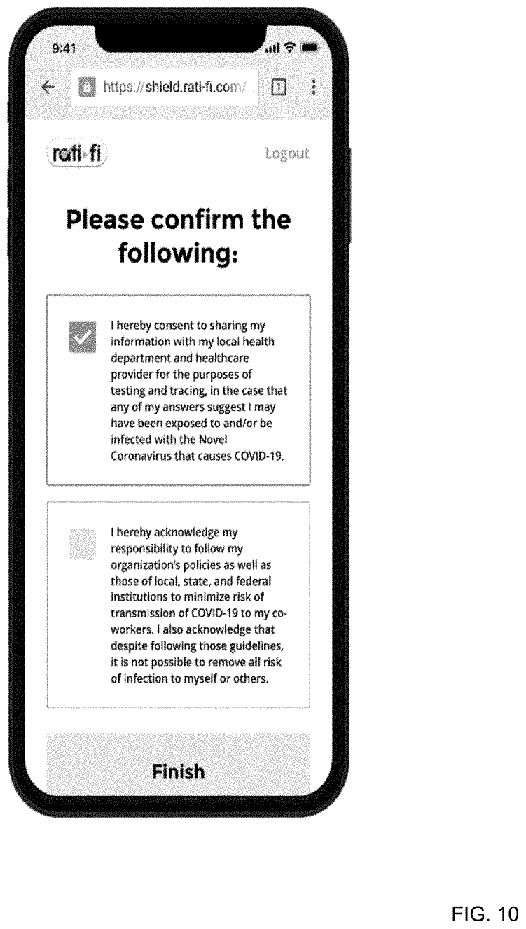

FIG. 11 Consent to Share . . . .

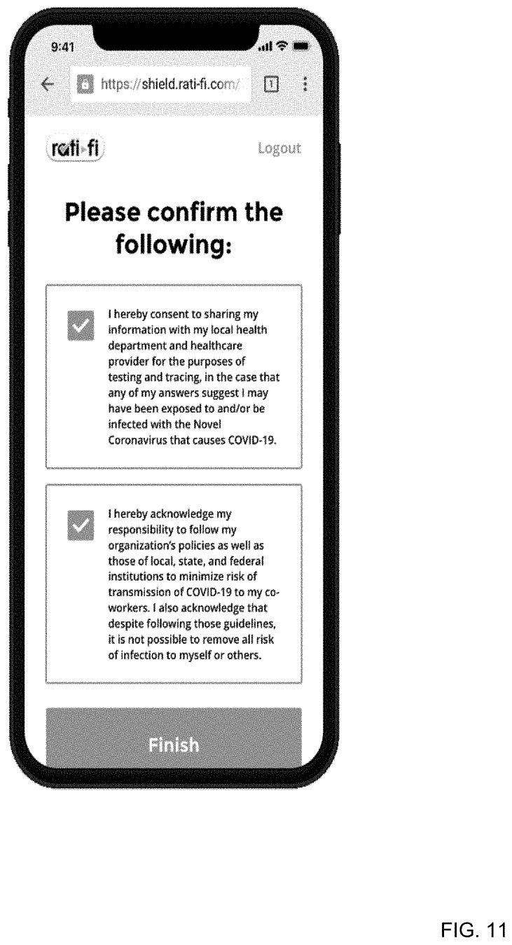

FIG. 12 Acknowledge Responsibility

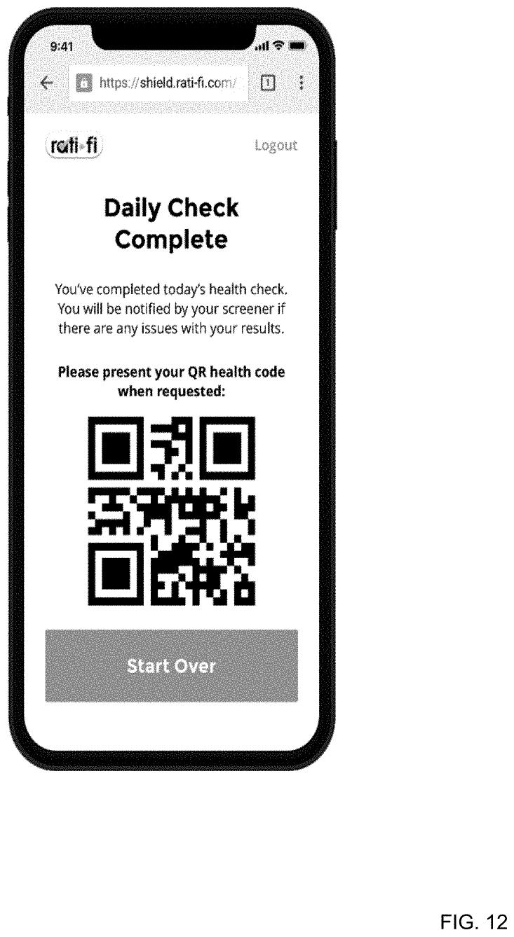

FIG. 13 Confirmations 1 & 2

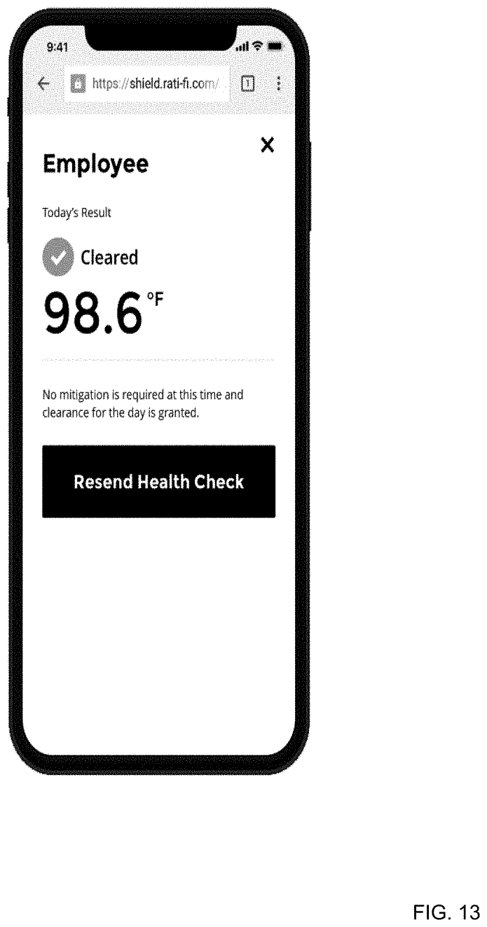

FIG. 14 Daily Check Complete . . . and Return of QR Code to Employee

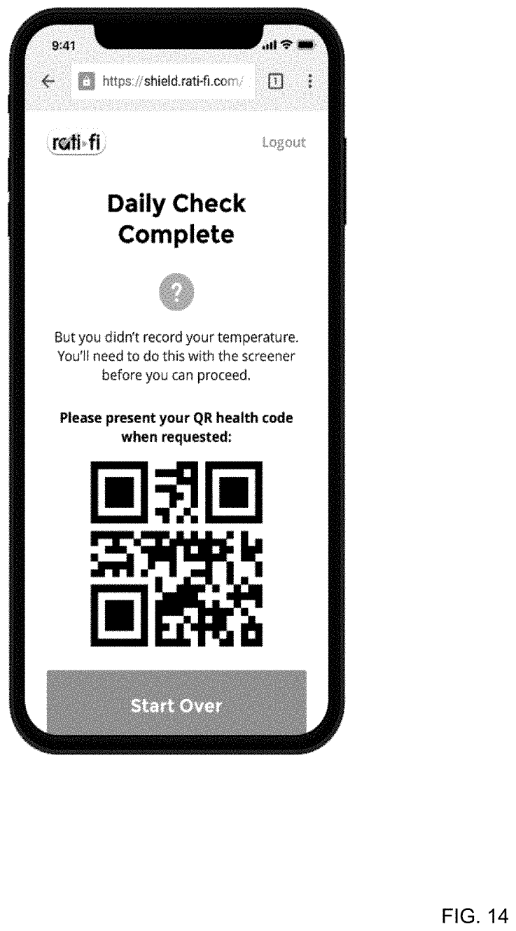

FIG. 15 Today's Result—Daily Check Complete or Missing Information Required; Daily Check Complete, You May Have Been Infected . . . See Supervisor

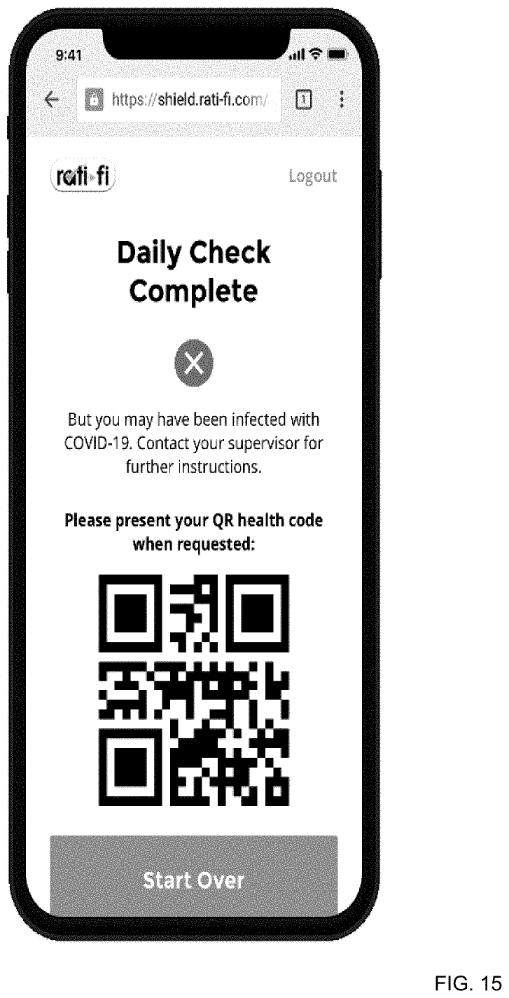

### BRIEF DESCRIPTION OF THE INVENTION

The present invention provides a system that combines employee historical health information with daily health information and, in some embodiments, with information regarding expected physical proximity of each worker at his/her workstation all in an electronic environment. The system combines historical and daily health information with the employee's physical position in the company where the employee performs the bulk of his work responsibilities, along with other physical locations the employee is expected to frequent within the company relative to job responsibilities e.g., break room, place/position on the line, etc. This information may be collected from each employee and provides internal contact tracing. The system also routinely requests specified consents from each employee pertaining to sharing and storing information in compliance with HIPAA, deletion or anonymization of data, and informed consent for tracking in company.as described below:

Specifically;
1. Employee confirms he has been informed that the electronic environment on which historical and new health information about employee will be shared is HIPAA compliant.
2. Employee confirms he has been informed that Company will delete or anonymize data or information that the employee submits within certain time limits.
3. Employee confirms he has been informed that his whereabouts while on company premises may be recorded by any of several means including movement of his employee badge for the purpose of contact tracing for use by Company solely for managing of spread of virus or other disease.
4. Employee confirms his understanding that the data so generated is used to determine trends, and hot spots within the company via physical location and/or commonalities of persons testing positive.

Having this information for all employees provides internal contact tracing capabilities. The system includes programming that asks the employee to respond to several questions pertaining to level of risk for transmission of an illness and stores and processes those responses. The system also seeks, receives, and records confirmation from the user of his understanding and agreement to comply with processes for mitigating/reducing transmission within the facility. The inventive system also documents employee informed consent for risk of disease exposure in the workplace. Finally, the system automatically performs daily tasks and facilitates reporting of test results, as needed. The system also documents the company's compliance with legally applicable disease or disaster mitigation guidelines on local, state, or federal levels.

As part of the system's operation, each employee is queried by the system to obtain employee confirmation that he permits company to share a specified, current threat, positive result within company policy. (Option to review policy should be provided here via link). The message may be delivered daily to each employee, and may contain a link. The link allows the user to enter the requested information and collect's the user's confirmations.

Specifically, the system may be as follows: employee comes to work and when he clocks-in, he may enter a kiosk which prevents others from seeing or hearing his responses. Other privacy measures may be employed in addition to, or instead of the kiosk, however, in one preferred embodiment of the system the kiosk is employed as a sort of booth where a single individual may enter and either verbally respond or respond via keyboard, or mouse, or touchscreen to several questions. He selects from several different languages (or tells the supervisor which language); the kiosk may present information via audible form, or visual on screen, or both. The employee answers several questions via selection from multiple choice answers to gather demographic and health-related data. The questions pertain to whether the worker has experienced any of several symptoms in the past 24 hours as means to determine if the worker is at low risk for transmission of a disease, such as COVID-19, which highly correlates with said symptoms. Additionally, the kiosk may query the same information regarding family members or persons with whom the worker lives. Additional questions may test whether the worker understands any restrictions in place pertaining to slowing transmission of the disease. Some embodiments of the present invention obtain employee confirmation of understanding of certain restrictions or requirements pertaining to slowing the spread of disease. When the worker has provided an answer to each of the questions, he is then presented with 3 different consents (see below) and is requested to respond to each of them before he can work. Finally, the worker's temperature is taken and recorded.

When the application determines the employee has completed the questionnaire, it will generate a result based on the entered information. The result may include a score or a category assigned by the application/system to the data submitted by the employee and, thereafter, the app will generate a QR code (or similar data and/or code) that indicates a status of the employee based on the information submitted by the employee via the question. That status will be used to determine whether the employee is permitted to work that day.

The information submitted by the employee/worker to the system or otherwise collected and submitted to the system is retained confidentially for a specified amount of time, and used for the purposes described herein. If the employer is obligated or elects to destroy the information provided by its employees, the system may be pre-set to routinely destroy information as it ages to a set time. For example, COVID19 often does not manifest certain symptoms until as long as two weeks from exposure. For that reason, auto-destruction may be set at, for example, 3-4 weeks to protect privacy while also gaining the maximum benefit of tracing re: Covid19. Of course, the system can be adjusted for other illnesses, as necessary, such as the seasonal flue; the questions asked and responses recorded may differ, triggers for action may differ, but the process, generally, would be similar. Further, the kiosk embodiment may be employed relative to a number of disease states and various physical business environments.

While the application may be used with a physical kiosk, it may optionally be used by employees at home before leaving their homes to come to work or immediately before entering their place of employment. In the at-home embodiment, the employee will receive a regular sms text or email which may be sent daily or on some other schedule. The text or email delivery format will be based on company policy and employee preference and comprises use of an imbedded weblink. The employee will complete the questionnaire via the application.

When the application determines the employee has completed the questionnaire, it will generate a result based on the entered information. The result may include a score or a category assigned by the application/system to the data submitted by the employee and, thereafter, the app will generate a QR code (or similar data and/or code) that indicates a status of the employee based on the information submitted by the employee via the questionnaire. The employee will show or submit the QR code to an electronic reader programmed to read the code and generate a status prior to entering the employer premises at the employee's place of work. The system may, optionally, further comprise means to store email addresses for all employees (or comprise means to generate a message to the employees seeking specific information). Where this function or feature is employed, a link may be transmitted with the message for the employee to send confirmation or to submit information requested.

Upon reading of the QR code (or other readable code) by the electronic reader (or a manually entered code) the system returns an indicator such as “CLEARED” “INCOMPLETE” OR “AT RISK.” It should be understood that the type of code or the type of reader may be one of several known in the art so long as the selected reader and code are usable together in the manner described herein. Employees who are indicated as cleared may enter. Those with incomplete screening will need additional screening, likely including at least a temperature reading and/or blood pressure and/or heart rate/or respiration. Those who are determined by the system to be at risk will be directed to employee health, their physician, or the health department for further evaluation depending on company policy.

In addition to employees, visitors to the business (scheduled and ad hoc) including contractors, customers/clients, or delivery people can be screened on a one time basis only (rather than scheduled, repetitive like employees) for screening before coming into a business. Also, schools, government buildings, sports and performance venues can use this application.

### Example 1

Employee opens kiosk, enters, and closes door to kiosk. Once the employee enters his name (or his employee badge or identification card is scanned by a reader to identify the employee) something similar to the following list of questions, confirmations, and consents will be presented, one at a time. The below series of questions is provided for example, and not for limitation. Presentation of the questions may be verbal, audible, in writing, or in a language selected by the employee. Once a response to the first confirmation, question or consent is received from employee and recorded by the system, then the next is presented:
INTRO: “Company has a policy that guides its disclosure and use of certain information about an employee's health for the protection of personal medical information of that employee and the protection of other workers. The company will employ the following company policies with regard to such information in its possession and or stored on company systems:
1. I understand that the company confirms that all personal medical data pertaining to an employee and stored in the system, including Covid-19 test results, are confidential and will be stored only via (all the words for the HIPAA acronym, and instructions where the employee can read the regulations pertaining to privacy re: test results if he wants to) a HIPAA compliant website, in compliance with HIPAA guidelines, and in accordance with Company policy, and I consent to such storage by Company and eventual destruction by Company of my personal medical data in its possession, if any.
2. I understand that the company confirms that all personal medical data pertaining to an employee and stored in the system, including Covid-19 test results, are confidential and will be shared only via (all the words for the HIPAA acronym, and where can the employee read the whole thing if he wants to) HIPAA compliant website, in compliance with HIPAA guidelines (available for review at______), and in accordance with Company policy (available for review at______), and I consent to such sharing by Company of my personal medical data in its possession.
3. I have been informed that Company policy is to delete personal medical information collected after X days, and/or to de-identify data, etc. and I consent to such destruction.
4. I have been informed and understand that my personal medical information may be used to slow the spread of the virus, and for contact tracing to understand or predict spread of the illness to or from others.
5. I have been informed that Company is implementing or has implemented a)______ b)______ and c)______ to mitigate and prevent spread of the corona virus.
6. I have been informed that Company may employ the data I submit here to mitigate spread of sickness among employees but that under no circumstance will information about my health be disclosed to______.
7. I recognize that even though the company is using these methods to prevent, mitigate, or prevent the spread of coronavirus within the Company, I could still become infected and ill.
8. I understand that I am obligated to honestly complete this questionnaire.

The system then evaluates the responses by comparing the responses selected by the employee with preset associated indicators; and records confirmation by employee of consent and of understanding of responsibility toward health and/or safety procedures regarding exposure risk. Thereafter, the system identifies at-risk individuals relative to responses provided by that individual as compared to the preset indicators, his temperature, his expected physical proximity to other workers while working, and the identities of the other workers working within a certain distance and/or other observations on his record. If, based on one or more of the aforementioned responses, data points, and proximity to other workers all as compared to standards and limitations pre-set in the system for this particular job and/or this particular employee's location while working, this employee is identified by the system as at-risk, the system generates and sends a pre-composed message (via email, or link) and instructs the employee to report to internal health services or to a supervisor or other reporting entity. The system may also trigger an alert regarding the at-risk result which is automatically sent to the individual's supervisor for immediate remediation. The system may generate a message to direct the employee to employee health services while creating and providing an alert message to the employee's supervisor. In situations where the employee is assigned to a team, or consistently works at particular position on the line next to the same co-workers, the system may be configured to generate and send an alert to the supervisor of that group of employees or that particular line (via intercom message, email, voicemail, or other means known). The system may be configured to contact each of these other workers and to generate requests for these workers to undergo additional health screening as a means to contain or reduce contamination.

High Level Functionality May Include, for Example:

User roles:
System administrator
Organization Administrator
Organization Screener
Registered user (employee/visitor)
Unregistered user

High level functionality Details/Outline:
Login
Manage Organization
Manage Users
Manage Surveys
Manage Locations
Send Survey Link
Screen Users
QR code lookup
Manage your Organizations
Take Survey
Register create account
Survey reminders
Other Resources (i.e., Centers for Disease Control, World Health Organization)
Take Survey
Hard code survey
Link to access and respond to survey
Multiple choice
Display result on user's device or link to open
New User:
Privacy Policy
Terms of Use
Manage Organizations edit user organization settings
Manage locations
Manage users
Send survey link
Survey reminders
May include QR Code lookup
Manage surveys

The system is expected to be comprised of bar code or QR readers, employee identification badges which may include bar codes or other electronically readable identification, database including multiple employees' employment information, job duties, general expected locations of the employee while working, assignment of the employee to any teams, a list of equipment the employee typically operates, and, in some embodiments, tracking equipment that tracks each employee's whereabouts within the company. Some or all of these tools may be employed as means to perform accurate contact tracing should an outbreak of illness occurs.

The system also may comprise and provide a kiosk for use by employees to comply with the employer's request for health information, said kiosk configured and equipped to electronically present a list of languages for selection by the employee and then present at least one question related to one or more of demographic characteristics and health conditions pertaining to the employee and/or the employee's family members in that language. The questions typically are expected to provide information that helps the company determine whether and which restrictions and measures may be needed in order to slow transmission of a specific disease, and to identify hot-spots relative to employee proximity. The responses are intended to be compiled by the system in such a way that the employer can identify sickness by physical location of the infected workers. This information allows the company to be more pro-active and to understand transmission routes so that mitigating systems and routines can be implemented. In short, the system will present to each said employee a selection of answers to each of said at least one questions regarding: symptoms, restrictions pertaining to slowing transmission of a disease; and the system will accept and record a response by said employee to each of said at least one question; further, the system will elicit, accept, and record at least one consent and/or confirmation provided by said employee; and record a body temperature of said employee. The system is further required to include routine data destruction routines, and to comply with data privacy rules, HIPAA regulations, and laws.

1. A health screening system comprising:
an electronic user interface configured to allow a user to enter demographic and health related data and provide consent to have the data at least one of stored in an electronic database, shared, and destroyed;
an application configured to generate a result based on the demographic and health related data and generate at least one of a QR code and a bar code which indicates a status related to the user, that status being related to whether the user is permitted to work; and
an electronic code reader configured to read the at least one of QR code and bar code, the health screening system further configured to return an indicator related to the status of the user.

2. The health screening system of claim 1, further comprising:
an electronic recording tool for recording a physical location of the user relative to time and record and combine historical health information of a plurality of employees, daily health information of the plurality of employees, and information pertaining to physical locations of the employees while performing job duties to assess employee health trends, and track and manage transmission of illness within the plurality of employees.

3. The health screening of claim 1, wherein the demographic and health related data comprises the user's responses to a series of questions.

4. The health screening system of claim 3, wherein the questions illicit information that correlates to level of risk for transmission of an illness.

5. The health screening system of claim 4, wherein the illness is COVID-19.

6. The health screening system of claim 1, wherein the employee further provides informed consent for provision of health data.

7. The health screening system of claim 1, wherein the employee further provides informed consent regarding understanding and agreement to comply with company processes for mitigation/reducing transmission within the facility.

8. The health screening system of claim 1, wherein the system documents employees' informed consent for risk of disease exposure.

9. The health screening system of claim 1, wherein the system generates reports, correlations, and trends of employees' health and test results relative to physical work station location.

10. The health screening system of claim 1, wherein the system documents the company's compliance with legally applicable disease or disaster mitigation guidelines.

11. A health screening and recording method comprising providing a kiosk for use by an employee, said kiosk configured and equipped to electronically;
present a list of languages for selection by the employee;
present at least one question related to one or more of demographic characteristics and health conditions pertaining to the employee and/or the employee's family members wherein at least one of said at least one question relates to restrictions intended to slow transmission of a specific disease;
present to said employee a selection of answers to each of said at least one questions regarding restrictions pertaining to slowing transmission of a disease;
accept and record a response by said employee to each of said at least one question;
elicit, accept, and record at least one consent by said employee;
record a body temperature of said employee; and
identify at-risk individuals and provide a notice to an at-risk individual and their supervisor.

12. The method of claim 11, further comprising storing of email addresses for employees.

13. The method of claim 11, further comprising a means to generate a message to employees seeking specific information.

14. The method of claim 11, further comprising scanning of an employee badge or identification card.

15. The method of claim 11, further comprising storing of responses collected in a HIPAA compliant database.

16. The method of claim 11, further comprising evaluation of responses.

17. (canceled)

18. The method of claim 11, further comprising notifying other employees within an at-risk employee's team.

19. The method of claim 11 wherein said at least one question and said selection of answers are presented in the language selected by the employee.
